(19) United States
(12) Patent Application Publication
(10) Pub. No.: US 2011/0250239 A1
(43) Pub. Date: Oct. 13, 2011

(54) PHARMACEUTICAL AND/OR COSMETIC COMPOSITION FOR TREATING THE SKIN

(75) Inventors: Hermann LUEBBERT, Leverkusen (DE); Montserrat Foguet ROCA, Leverkusen (DE); Mirella GWAREK, Koeln (DE)

(73) Assignee: BIOFRONTERA BIOSCIENCE GMBH, Leverkusen (DE)

(21) Appl. No.: 13/081,737

(22) Filed: Apr. 7, 2011

Related U.S. Application Data
(60) Provisional application No. 61/322,524, filed on Apr. 9, 2010.

(30) Foreign Application Priority Data
Apr. 9, 2010 (DE) 20 2010 004 750.1

(51) Int. Cl. A61K 36/00; A61K 36/29; A61K 36/484; A61K 36/21; A61K 36/68; A61P 29/00; A61K 36/54; A61K 36/87; A61P 17/10; A61P 17/04; A61P 17/06; A61P 17/00; A61K 9/107; A61K 36/62

(52) U.S. Cl. 424/400; 424/725; 424/757; 424/738; 424/739; 424/766

(57) ABSTRACT

The present invention concerns the pharmaceutical and cosmetic application of biocolloids, which originate from the mixture of a nanoemulsion and at least one water-soluble plant extract such as an extract from Mahonia aquifolium, wherein the biological effect of the plant ingredients is enhanced by the composition although the nanoemulsion does not function as a carrier system. The biocolloids are especially for the treatment and care of the skin.

The present invention concerns a pharmaceutical and/or cosmetic composition comprising a biocolloid comprising a plant extract, e.g. from Mahonia aquifolium, which is particularly suitable for treating the skin, and a method for preparing the same.

No other human organ has such a high number of pathological changes as the skin because the skin as a boundary organ of the human body is exposed to a wide variety of influences from inside and outside. The number of skin diseases has considerably increased in the past decades. For this reason new pharmaceutical and cosmetic compositions for treating the skin are desirable.

Nanoemulsions can be used as carrier systems for active substances, in particular, for active substances which are insoluble in water. Nanoemulsions are a colloidal system. Other colloidal systems are for example micelles, liposomes, virosomes, nanosuspensions, microemulsions or polymer solutions. Microemulsions are aqueous dispersions of homogeneous particles in the microrange which are composed of a lipid core that is surrounded by surfactant and cosurfactant monolayers. The physical and chemical properties of nanoemulsions are similar to those of microemulsions but they differ in their particle size. Nanoemulsions are characterized by an average particle size (average diameter) of less than 500 nm and often also less than 200 nm and a narrow monodisperse particle size distribution.

EP 0 956 853 describes nanodispersions comprising a) a membrane-forming molecule e.g. soya lecithin, b) a co-emulsifier, c) a lipophilic component e.g. capryl and/or capric triglyceride (Myglyol 812 or Myritol 318) and optionally d) ethanol. These nanodispersions are used in pharmaceutical formulations as transport vehicles for active pharmaceutical ingredients.

WO 2008/077641 discloses a nanoemulsion comprising a) at least one aqueous component and b) a carrier which comprises i) at least one lipophilic component, ii) at least one surfactant and iii) at least one alcohol, wherein the at least one alcohol has at least three carbon atoms. The at least one alcohol is preferably isopropyl alcohol and/or 1-propyl alcohol. The nanoemulsion described in WO 2008/077641 has an optimal, small average particle size with a narrow particle size distribution. The small particle size allows one to obtain a nanoemulsion with high stability and good penetration into cells and tissue.

Mahonia aquifolium is an evergreen shrub from the Mahonia genus in the family of the barberry plants. It originally comes from Western North America where it has been used for many centuries by the natives of North America for treating psoriasis and eczema. The roots and the stem bark contain berberine as well as various other alkaloids from the group of isoquinoline alkaloids such as for example magnoflorin, berbamine, palmatine, jatrorrhicine, bervulcine, columbamine and oxyacanthine. These alkaloids are known for their anti-inflammatory, antioxidative and antiproliferative properties.

WO 2005/027945 describes that berberine and other alkaloids mentioned above interact reversibly with DNA which results in an inhibition of replication and transcription. Furthermore, WO 2005/027945 describes that the alkaloids which are extracted from the Mahonia aquifolium extract exhibit an anti-proliferative activity on keratinocytes. Since hyperproliferation of keratinocytes is one of the main symptoms of psoriasis, the control of hyperproliferation is one therapeutic approach for psoriasis. Furthermore, the effectiveness of protoberberine alkaloids is narrowly linked to lipoxygenase inhibition. Additionally, active ingredients from Mahonia aquifolium are leukotriene B4 (LTB4) inhibitors (it is assumed that LTB4 is involved in inflammations) and inhibitors of 5-hydroxy-arachidonic acid (5-HETE). Thus, the extract from Mahonia aquifolium exhibits a similar efficacy as dithranol which is one of the most important drugs for treating psoriasis. Furthermore, it was found that some alkaloids in the extract from Mahonia aquifolium have anti-inflammatory properties due to the inhibition of lipoxygenase. Oxidation products which are derived from lipoxygenase play a mediator role in inflammations in other biological systems.

WO 2005/027945 discloses a composition for treating the skin which comprises a Mahonia aquifolium extract in a liposome transport system. Treatment of psoriasis, eczema, dermatitis and other dry skin conditions with this composition is described.

However, the use of liposome transport systems has several disadvantages. On the one hand, it is known that liposomes have a low inclusion stability which is due to slow diffusion of hydrophilic active ingredients from the aqueous interior through the lipid membranes of the liposomes. As a result the concentration of active ingredient decreases in the liposomes which consequently leads to a reduced transport of active ingredient to the target site. Furthermore, it is known that liposomes have a low storage stability which is due to the fact that the liposomes for example burst or fuse.

Hence an object of the present invention was to provide a pharmaceutical and/or cosmetic composition comprising a plant extract from Mahonia aquifolium which has a higher stability and in particular enables a better effect of the components of the Mahonia aquifolium extract.

The object was achieved by providing a pharmaceutical and/or cosmetic composition comprising a biocolloid comprising a nanoemulsion and at least one water-soluble plant extract.

The composition according to the invention comprising a biocolloid can be formed or made by mixing a plant extract, preferably at least one extract from Mahonia aquifolium, and a nanoemulsion. By biocolloid is meant a colloidal system, which is generated by mixing at least one nanoemulsion and at least one plant extract. The biocolloid according to the invention in particular acts synergetically on biological systems compared to the single components, although the employed nanoemulsions are no carrier systems for the active ingredients derived from plants. Further, the mixture of several different plant extracts with a nanoemulsion is preferred.

The compositions according to the invention comprise a water-soluble plant extract. Due to the water-solubility of the extract the substances contained in the extract do not penetrate the colloids of the nanoemulsion. Thus, the nanoemulsion does not act as a carrier system as in other products. This can for example be seen that by mere addition of the extract the size of the single micelles does not change. In conventional systems nanoemulsions were often employed as carrier systems for water-insoluble or hydrophobic agents. Thereby interactions between the micelles of the nanoemulsion, which have an oil phase at the inside and lipids at the outside, and the water-insoluble and hydrophobic agents, respectively, are formed, so that the nanoemulsion serves as a carrier system. In contrast thereto, water-soluble plant extracts are employed according to the present invention, which do not form interactions with the micelles of the nanoemulsions.

The inventors have found that, nevertheless, due to combined action of the components of the herein described composition, the biological effect of the substances contained in the extract on skin cells (keratinocytes) is surprisingly increased. Furthermore, it was found that the composition according to the present invention reduces antigen presentation on the skin cells considerably stronger compared to addition of the extract alone. This can result in stronger reduction of inflammatory processes than without the composition according to the present invention. Furthermore, it turned out that the composition according to the present invention is more stable with regard to long term stress conditions (e.g. increased temperature) than the formulation described in WO2005/027945.

The water-soluble plant extract used in the composition according to the present invention is preferably an extract from Mahonia aquifolium, an extract from Glycyrrhiza glabra, an extract from Salicornia herbacea, a leaf extract from Plantago lanceolata, a blossom extract from Nymphea alba, a bark extract from Cinnamomum zeylanicum or a leaf extract from red wine or a combination thereof.

The nanoemulsion employed for the preparation of the composition according to the invention can generally comprise any components. However, it preferably comprises a) at least one aqueous component and b) a carrier comprising i) at least one lipophilic component, ii) at least one surfactant and iii) at least one alcohol.

The nanoemulsion preferably comprises an aqueous component in an amount of 50% by weight to 98% by weight, more preferably from 70% by weight to 95% by weight and most preferably from 88% by weight to 92% by weight based on the total weight of the nanoemulsion. This specific composition advantageously results in a nanoemulsion which is very well tolerated by the skin and which does not lead to a sticky feeling during the application on the skin. This can be achieved inter alia by keeping the proportion of other components, for example surfactants and lipophilic components relatively low.

The aqueous component of the nanoemulsion preferably comprises a buffer system e.g. a weak buffer system with a low salt content. A buffer system is preferred which has a salt content of 5 nM to 30 nM, in particular a phosphate buffer system. A buffer system with a salt content of 7 nM to 20 nM is more preferred, in particular a phosphate buffer system. A buffer system with a salt content of 8 nM to 12 nM is most preferred, in particular a phosphate buffer system. The pH of the buffer, preferably a phosphate buffer, is preferably in a range of pH 4 to pH 8, more preferably of pH 5 to pH 7 and most preferably of pH 5.5 to pH 6.5. The water which is used for the preparation of the buffer, in particular a phosphate buffer is preferably sterile deionized water and/or water for injection purposes, more preferably water for injection purposes.

The nanoemulsion comprises component i) of the carrier i.e. the lipophilic component preferably in an amount of 0.1% by weight to 15% by weight, more preferably of 1% by weight to 8% by weight and most preferably of 3% by weight to 4% by weight based on the total weight of the nanoemulsion. Component ii) of the carrier i.e. the surfactant is preferably present in an amount of 1% by weight to 30% by weight, more preferably of 2% by weight to 15% by weight and most preferably of 4% by weight to 6% by weight based on the total weight of the nanoemulsion. Component iii) of the carrier, i.e. the alcohol is preferably present in an amount of 0.1% by weight to 10% by weight, more preferably of 0.5% by weight to 5% by weight and most preferably of 1% by weight to 2% by weight based on the total weight of the nanoemulsion.

As component i) of the carrier, i.e. the at least one lipophilic component, can for example a lipid, a vegetable oil and/or an animal oil be used. Suitable lipids for the nanoemulsion of the composition according to the invention are physiologically acceptable lipids in particular ceramides, glycerides (acylglycerine) such as mono-, di- and triglycerides and/or mixtures thereof. Preferably the lipophilic component comprises a triglyceride or a mixture of triglycerides, whereas as triglycerides preferably capryl- and/or capric-triglycerides, more preferably miglyol (such as for example Miglyol 812 or Myritol 318 obtainable for example from Henkel) are used. Suitable vegetable and animal oils are for example coconut oil, sunflower oil, soya bean oil, peanut oil, rapeseed oil, fish oil and/or cetaceum.

As component ii) of the carrier, i.e. the at least one surfactant, can for example a membrane-forming surfactant or/and an O—W-emulsion forming surfactant be employed. Membrane-forming surfactants comprise for example phospholipids, lysophospho-lipids, ceramides as well as mixtures thereof, particularly preferable are phospholipids. In case a phospholipid is employed, the phospholipid is preferably lecithin or cephalin, which may be for example derived from soya beans or chicken eggs. A tenside which comprises soya lecithin is most preferred.

In case the surfactant comprises lecithin, the latter has preferably a phosphatidyl choline content of at least 80% by weight, more preferably of at least 90% by weight and most preferably of at least 94% by weight. The inventors have found that the quality of the lecithin which is essentially determined by its phosphatidyl choline content plays an important role for the size of the particles of the nanoemulsion. The higher the phosphatidyl choline content of the lecithin, the smaller is the size of the particles of the nanoemulsion.

Anionic, non-ionic, cationic and/or amphoteric surfactants as well as block copolymers are suitable as the O—W-emulsion-forming surfactant. It was found that non-ionic surfactants are particularly preferred. Suitable anionic surfactants are soaps, alkyl benzene, sulfonates, alkane sulfonates, alkyl sulfates and/or alkyl ether sulfates. Suitable cationic surfactants are quarternary ammonium compounds, preferably with one or more hydrophobic groups (e.g. cetyltrimethylammonium bromide and cetyltrimethylammonium chloride) and/or salts of long chain primary amines. Suitable amphoteric surfactants are N-(acyl-amidoalkyl)betaine, N-alkyl-β-aminopropionate and/or amine-N-oxide. A suitable block copolymer is for example propylene oxide.

Suitable non-ionic surfactants comprise for example compounds selected from the group consisting of fatty acid alcohol polyglycol ethers, alkylphenol polyglycol ethers, alkylpolyglucosides, fatty acid glucamides, fatty acid polyglycol ethers, ethylene oxide propylene oxide block copolymers, polyglycerol fatty acid esters, fatty acid alkanolamides and (ethoxylated) sorbitan fatty acid esters (sorbitan). According to the present invention, surfactants of the polyoxyethylene type (polyoxyethylene surfactants) are preferred, such as for example ethoxylated sorbitan fatty acid esters. More preferably, the polyoxyethylene surfactant is polyoxyethylene sorbitan monooleate and most preferably Polysorbate 80.

In one preferred embodiment of the present invention the nanoemulsion comprises a membrane-forming surfactant and an O—W-emulsion-forming surfactant, such as for example a combination of a phosholipid and a non-ionic surfactant. The weight ratio of the amount of membrane-forming surfactant:O—W-emulsion-forming surfactant is, in particular, in the range of 0.1:1 to 10:1, preferably of 0.2:1 to 0.8:1, more preferably of 0.4:1 to 0.6:1.

As component iii) of the carrier, i.e. the at least one alcohol can for example an alcohol with 1 to 20 carbon atoms be employed, wherein an alcohol with at least two carbon atoms has been found to be advantageous. A preferred alcohol is an alcohol with two to ten carbon atoms, more preferably two to seven carbon atoms, even more preferably two to five carbon atoms and most preferably two to three carbon atoms, wherein the alcohol is in particular preferably an aliphatic saturated alcohol. Particularly suitable alcohols with five carbon atoms are 1-pentanol and 3-methyl-1-butanol. Suitable alcohols with four carbon atoms are 1-butanol (n-butanol), 2-butanol (sec-butanol), 2-methyl-1-propanol (iso-butanol) and 2-methyl-2-propanol (tert.-butanol), while as alcohols with three carbon atoms, in particular 1-propanol and 2-propanol (iso-propanol) can be used. In the most preferred embodiment the alcohol is ethanol or 2-propanol.

The average diameter of the emulsified micelles or particles in the nanoemulsion (nanosomes) is generally 1 nm to 1000 nm, preferably 1 nm to 500 nm, more preferably 10 nm to 200 nm, even more preferably less than 100 nm and in particular up to 90 nm, preferably up to 70 nm, yet more preferably 10 nm to 50 nm and most preferably 15 nm to 35 nm. The size distribution of the micelles or nanoparticles is preferably monodisperse and corresponds to a Gaussian distribution. The diameter of the emulsified micelles or particles of the nanoemulsion can be measured by methods for determining the particle size distribution such as for example dynamic light scattering (DLS) (also referred to as photon correlation spectroscopy (PCS)). The statistical analysis of the micelle distribution and particle distribution, respectively, can be carried out by a method which is called particle count-weighted distribution.

The amount of nanoemulsion in the pharmaceutical and/or cosmetic composition is preferably 20% by weight to 90% by weight, more preferably 30 to 70% by weight and most preferably 35 and 45% by weight based on the total weight of the composition.

The water-soluble plant extract can for example be obtained by an extraction from the particular plant by using a suitable extraction agent. For this purpose any components of the particular plant can in general be extracted, which seem suitable for the purpose of the present invention to the person skilled in the art such as for example roots, bark, twigs, leaves and/or blossoms. According to the present invention, preferably, dried components of the particular plant are extracted, wherein in particular a mixture of water and an alcohol can be used as extraction agent. For the preparation of a plant extract from Mahonia aquifolium preferably branch and twig bark, as well as twig tips are employed as dried components. Ethanol is a particularly preferred alcohol for the extraction. Preferred ratios for the mixture of water and alcohol contain at least 50% alcohol. The use of 65% to 90% alcohol is preferred. Further preferred extraction methods comprise the use of glycerine or glycol as extraction agent.

The preparation of a plant extract from Mahonia aquifolium can for example be performed as described in WO 2005/027945. In this process the dried plant components from Mahonia aquifolium are heated for three hours under pressure and while stirring in a mixture of alcohol and water. Subsequently the reaction mixture is allowed to cool for 24 hours. These two steps are repeated at least twice. After removing the water and alcohol and after filtration, the plant extract from Mahonia aquifolium is obtained.

Alternatively, a mother tincture according to the homeopathic pharmacopoeia can be prepared by a five day incubation of one part plants with ten parts 62% ethanol at a temperature which does not exceed 20° C. The extraction process can be enhanced by percolation or maceration.

Further, the extraction agent such as the alcohol can be withdrawn from the extract or the mother tincture, for example by evaporation or drying, respectively, and subsequent dissolving in water.

The plant extract from Mahonia aquifolium obtained by the above-mentioned processes contains alkaloids in particular isoquinoline alkaloids which have anti-inflammatory, antioxidative and antiproliferative properties. The plant extract from Mahonia aquifolium used in the present invention preferably contains between about 0.5 mg to 30 mg isoquinoline alkaloids per gram extract, more preferably about 1 mg to 20 mg isoquinoline alkaloids per gram extract and most preferably about 2 mg to 10 mg isoquinoline alkaloids per gram extract. The plant extract from Mahonia aquifolium used in the present invention preferably contains berberine particularly preferably between about 0.5 mg to 3 mg berberine per gram extract, more preferably about 1 mg to 2.5 mg berberine per gram extract and most preferably about 1.3 mg to 1.8 mg berberine per gram extract.

The amount of the water-soluble plant extract, in particular an extract from Mahonia aquifolium, in the pharmaceutical and/or cosmetic composition of the present invention is preferably 1% by weight to 20% by weight, more preferably 5% by weight to 18% by weight and most preferably 7% by weight to 15% by weight based on the total weight of the composition. At least 0.0015% by weight berberine and at most 0.03% by weight berberine are preferably present in the total composition.

The pharmaceutical and/or cosmetic composition of the present invention additionally comprises an aqueous component in an amount of 20% by weight to 60% by weight, more preferably in an amount of 30% by weight to 50% by weight and most preferably in an amount of 35% by weight to 45% by weight based on the total weight of the composition. The water in the nanoemulsion is not included here. In a preferred embodiment of the present invention the aqueous component is water. The total water content in the composition by including the aqueous component and the nanoemulsion component is thus preferably 40% to 95% by weight, in particular 60% to 85% by weight.

The pharmaceutical and/or cosmetic composition of the invention preferably additionally comprises at least one filling agent preferably in an amount of 0.01% by weight to 4% by weight, more preferably in an amount of 0.5% by weight to 2% by weight based on the total weight of the composition. The filling agent is sodium carbomer in a preferred embodiment.

The pharmaceutical and/or cosmetic composition of the present invention can additionally contain a preservative in an amount of preferably 0% by weight to 2% by weight, more preferably of 0.5% by weight to 1% by weight based on the total weight of the composition. Suitable preservatives for the present invention can for example be phenoxy-ethanol, 1,2-propylene glycol, methyl parahydroxybenzoate and/or propyl parahydroxybenzoate. In a preferred embodiment the preservative is phenoxy-ethanol.

In a further preferred embodiment the pharmaceutical and/or cosmetic composition of the present invention can preferably contain further plant extracts. In preferred embodiments extracts having antioxidative or anti-inflammatory effects can be added. Preferably is for example the addition of 0% by weight to 5% by weight, more preferably 0.005% by weight to 1% by weight of a green tea extract. As further anti-inflammatory agent can preferably a camomile extract in an amount of 0% by weight to 5% by weight, more preferably in an amount of 0.02% by weight to 1% by weight be contained. In a particularly preferable embodiment both extracts are added to the biocolloid.

In a preferred embodiment the pharmaceutical and/or cosmetic composition according to the invention comprises 0.1% by weight to 3% by weight lecithin, preferably with a phosphatidyl cholin content of 94% in the nanoemulsion. Further, the nanoemulsion can comprise 0.5% by weight to 2% by weight triglycerides which can preferably be capryl-/capric triglycerides. Furthermore, the nanoemulsion can comprise 0.5% by weight to 2% by weight surfactant, preferably a polyoxyethylene surfactant which can preferably be Polysorbate 80. Further, the nanoemulsion can contain 0.1% by weight to 3% by weight alcohol, preferably an alcohol which comprises two to three carbon atoms preferably isopropyl alcohol. Furthermore, the nanoemulsion contains a phosphate buffer system with a salt content of 1 to 7 nM and 50% by weight to 80% by weight water, preferably sterile, deionized water and/or water for injection purposes.

The composition according to the invention can further contain 7% by weight to 15% by weight plant extract from Mahonia aquifolium.

Furthermore, the composition according to the invention can contain 0.001% by weight to 4% by weight filling agent where in particular sodium carbomer is preferred as filling agent. Further, the composition according to the invention can contain 0.5% by weight to 2% by weight preservative where in particular phenoxyethanol is preferred as preservative. The composition according to the invention can optionally contain 0% by weight to 5% by weight of a further anti-inflammatory agent, where the further anti-inflammatory agent is preferably of plant origin. In particular camomile extract is preferred as an anti-inflammatory agent. The composition according to the invention can optionally contain 0% by weight to 5% by weight of an oxidation-inhibiting agent, where the oxidation-inhibiting agent is preferably of plant origin. Green tea extract is especially preferred as oxidation-inhibiting agent.

Further, the composition according to the invention can contain 0% by weight to 20% by weight further additives such as for example glycerol, xanthan gum, disodium EDTA, tocopheryl acetate, propylene glycol, bis-PEG/PPG-14/14 dimethicone, cyclopentasiloxane and cetyl alcohol. The remainder of the composition according to the invention can be water, preferably sterile, deionized water and/or water for injection purposes. The weight percentages of this and the previous two sections are based on the total weight of the composition. By addition of additives, the size of the micells which are contained in the biocolloid solution can be adjusted. In a preferred preparation they have a diameter of from 20 to 1.000 nm, further preferred 20 to 450 nm.

In a particularly preferred embodiment the pharmaceutical and/or cosmetic composition according to the invention has the following composition (based on the total weight of the composition): 0.1% by weight to 2% by weight lecithin with at least 94% of phosphatidyl cholin, 0.5% by weight to 2% by weight Polysorbate 80, 0.5% by weight to 2% by weight capryl-/capric triglycerides, 0.1% by weight to 2% by weight isopropyl alcohol, 0.001% by weight to 0.1% by weight disodium phosphate dihydrate, 0.01% by weight to 0.3% by weight sodium dihydrogen phosphate dihydrate and 30% by weight to 40% by weight deionized water as the nanoemulsion and 7% by weight to 15% by weight plant extract from Mahonia aquifolium, 20% by weight to 40% by weight deionized water, 2% by weight to 5% by weight glycerol, 0.1% by weight to 1% by weight xanthan gum, 0.5% by weight to 3% by weight sodium carbomer, 0.01% by weight to 0.3% by weight disodium EDTA, 1% by weight to 4% by weight 1,2-propylene glycol, 0.01% by weight to 0.5% by weight tocopheryl acetate, 0.1% by weight to 2% by weight phenoxyethanol, 0.5% by weight to 3% by weight bis-PEG/PPG-14/14 dimethicone, 0.2% by weight to 1% by weight cyclopentasiloxane, 0.5% by weight to 3% by weight cetyl alcohol, 0% by weight to 1% by weight camomile extract and 0% by weight to 0.01% by weight green tea extract.

In another embodiment the composition according to the invention can contain an appropriate amount of further additives such as for example scents or moisturizer cream. Since only a relatively small amount of emulsifiers is used, the composition has a mild and gentle action on the skin of the user.

The invention concerns pharmaceutical as well as cosmetic applications. For an intended cosmetic use of the final product, it is possible to add further agents such as for example emollients, dyes, scents, gelling agents, thickeners, sun screen agents and suchlike. These additives improve the use of the final product especially for topical cosmetic applications. The addition of the above-mentioned agents should be selected taking care that the external appearance of the product is not adversely affected e.g. with regard to homogeneity. The cosmetic composition can be present inter alia in the form of a cream, milk, lotion or a gel, serum, hair tonic or a liquid spray. If UV absorbing components are added to the composition according to the invention, one or more sun screen agents such as for example benzophenones, avobenzones, cinnamic acid derivatives, salicylates, titanium oxide and suchlike can be used.

For an intended pharmaceutical use of the final product it is also possible to add further agents such as for example emollients, dyes, scents, gelling agents, thickeners, sun screen agents and suchlike. These additives improve the use of the final product especially for topical pharmaceutical applications. Other active agents can also be added for a pharmaceutical use such as for example agents for erasing age spots, keratodermas and wrinkles, analgesics, anaesthetics, anti-acne agents, antimycotics, virostatic agents, anti-dandruff agents, antiemetic agents, agents against travel sickness, antiperspirants, antiasthmatic agents, anti-aging agents, bronchodilators, antihistamine agents, depigmentation agents, vitamins, corticosteroids, hormones, retinoids such as for example retinoic acid and retinol, topic cardiovascular agents, clotrimazole, ketoconazole, miconazole, griseofulvin, hydroxyzine, diphenylamine, pramoxine, lidocaine, procaine, mepivacaine, monobenzone, antibiotics such as for example erythromycin, tetracyclin, clindamycin, kanamycin, minocyclin and meclocyclin, hydroquinone, naproxen, ibuprofen, theophylline, cromolyn, albuterol, topical steroids such as for example hydrocortisone, hydrocortisone-21-acetate, hydrocortisone-17-valerate and hydrocortisone-17-butyrate, betamethasone valerate, betamethasone diproprionate, triamcinolone acetonide, fluocinonide, clobetasol proprionate, benzoyl peroxide, crotamiton, propranolol, promethazine, vitamin A palmitate, vitamin E acetate and mixtures thereof. The pharmaceutical composition can be present inter alia in the form of a cream, milk, lotion or a gel, serum, hair tonic or liquid spray. If a sunscreen agent is added to the composition according to the invention, it is possible to add one or more sunscreen agents such as for example benzophenones, avobenzones, cinnamic acid derivatives, salicylates and suchlike.

In a further embodiment the composition according to the invention can additionally comprise cosmetic and/or pharmaceutically acceptable additives and/or auxiliary agents, especially substances which are commonly used in the cosmetic industry or pharmaceutical industry. Examples of such substances are buffers, stabilizers, additional emulsifiers, thickeners etc. Moreover, the composition according to the invention does not have any components which are pharmaceutically or cosmetically unacceptable and the composition preferably also does not have any components which for example cause skin irritations. In addition to the carrier substances which have already been mentioned, the composition can contain further adjuvants and/or additives which are acceptable for dermatological and cosmetic applications and are well-tolerated. The pharmaceutical and/or cosmetic composition can inter alia be present in the form of a cream, milk, lotion or a gel, hair tonic, serum or liquid spray.

A further aspect of the present invention is a process for the preparation of the pharmaceutical and/or cosmetic composition according to the present invention comprising the following steps:

a) providing an aqueous component
b) providing a carrier comprising at least one lipophilic component, at least one surfactant and at least one alcohol, wherein the alcohol comprises preferably at least two carbon atoms
c) mixing the aqueous component of step a) with the carrier of step b), whereby a nanoemulsion is formed
d) adding an aqueous plant extract, in particular a water-soluble plant extract from Mahonia aquifolium to the mixture obtained in step c) prior to and/or after the formation of said nanoemulsion and
e) optionally adding further additives and/or excipients to the mixture obtained in step d).

Preferably the plant extract is added in step d) to the mixture obtained in step c) after the formation of said nanoemulsion. Regarding preferred embodiments of the employed aqueous component, carrier and aqueous plant extract, respectively, in the above process, reference is made to the statements with regard to the description of the composition according to the invention.

The properties of the composition according to the invention can be influenced in particular by adjusting the temperature and the mixing conditions, respectively, in step c) of the above described process. Step c) is preferably carried out at a temperature in a range between 20 to 80° C., more preferably between 30 to 70° C. and most preferably between 50 to 60° C., whereby all ingredients are heated up to this temperature beforehand. The mixing of the aqueous component and the carrier is preferably performed by gentle homogenization and more preferably by intensive homogenization, which may be carried out, e.g. by a commercially available homogenizer. The container and the mixer should also be optimized in order to avoid foam formation to allow a very fast and homogenous mixing of the components (within a few seconds). In contrast, the homogenization does not require high shear devices, such as sonicators or high pressure homogenizers. The particular plant extract, as well as optionally present additives and/or carriers may be added prior to and/or after the homogenization of the nanoemulsion, preferably after the homogenization.

Generally, the process is carried out at a temperature at which the nanoemulsion can be formed, as mentioned above, and at which the individual components of the composition, in particular the aqueous plant extract are adequately stable for the subsequent steps. In general, it has been found that a temperature range of from about 5 to 30° C. is advantageous, preferably of from about 10 to 25° C. and more preferably of from about 15 to 23° C. The processing of the additives and/or excipients, which are optionally mixed together and homogenized first and may only be added after that to the composition, can also be carried out at higher temperatures, e.g. at a temperature of up to about 80° C.

The herein described processing method is preferably performed under aspetic conditions, e.g. by using a laminar flow hood, by excluding air, e.g. by means of applying a vacuum and/or under a protective gas atmosphere and/or by excluding light. Especially for pharmaceutical applications, care is to be taken that the final product is sterile, e.g. by using sterile starting materials, by applying sterile processing conditions and/or by means of a sterilization step following preparation of the composition.

The composition according to the invention utilizes the advantages of the nanoemulsion described above which provides a translucent and transparent vehicle so that the active ingredients from the plant extract from Mahonia aquifolium can penetrate into the skin and display their anti-inflammatory, antioxidative and antiproliferative action there. The pharmaceutical and/or cosmetic composition according to the invention is thus particularly useful for topical application on the skin and scalp.

The composition according to the invention can be used for topical application. The composition according to the invention is preferably applied topically on the skin and scalp, respectively. In a particularly preferred embodiment the composition according to the invention is used for the prevention and/or treatment of skin irritations and skin diseases, wherein the herein used term “treatment” also comprises a skin regeneration.

In a special embodiment the pharmaceutical and/or cosmetic composition is used for the prevention and/or treatment of dermatological diseases. Dermatological diseases are understood as those which represent diseases of the skin and mucous membranes such as for example dermatitis, eczema and psoriasis. In a preferred embodiment the composition according to the invention can be used for the prevention and/or treatment of atopic dermatitis and/or psoriasis.

In a further preferred embodiment the pharmaceutical and/or cosmetic composition is used for the prevention and/or treatment of skin irritations, for example for the prevention and/or treatment of itching and/or irritated skin. “Skin irritations” in connection with this invention are understood as a chronic or non-chronic condition of the skin in which the skin is dry and itches. It is also understood to include inflamed, red areas which sometimes have blisters and are wet. Between the red and inflamed areas, sections of the skin may appear to be normal or have chronic eczemas with dry, thickened and itching areas.

In further preferred embodiments of the present invention, the herein described composition can be used for the prevention and/or treatment of acne and/or rosacea and for the prevention and/or treatment of insect stings, respectively.

In a preferred embodiment the composition according to the invention is used regularly for topical application on the skin. Regular use in connection with the invention is understood to mean that the composition according to the present invention is applied to the skin at regular intervals over an unspecified period e.g. once to three times daily.

The invention is further directed to a method for the prevention or treatment of skin irritations or skin diseases, comprising administration of an effective amount of a pharmaceutical or cosmetic composition as described herein to a subject in need thereof.

An effective amount, in particular, is an amount which results in an improvement of the condition to be treated. According to the invention, in particular, atopic dermatitis, psoriasis, itching, irritated skin, acne, rosacea or insect stings can be treated using the composition described herein.

The following figures and examples are intended to further illustrate the present invention:

### DESCRIPTION OF THE FIGURES

FIG. 1 shows the change of the size distribution of micells in a nanoemulsion measured with a nanosizer (Malvern) upon addition of a water-soluble plant extract.

(A): Size distribution of micells in the nanoemulsion before additon of an extract from Mahonia aquifolium.
(B): Size distribution of micells in the nanoemulsion after additon of an extract from Mahonia aquifolium in a 1:10 ratio.

FIG. 2 shows the kinetics of the redness reduction of injured skin after application of a composition according to the invention.

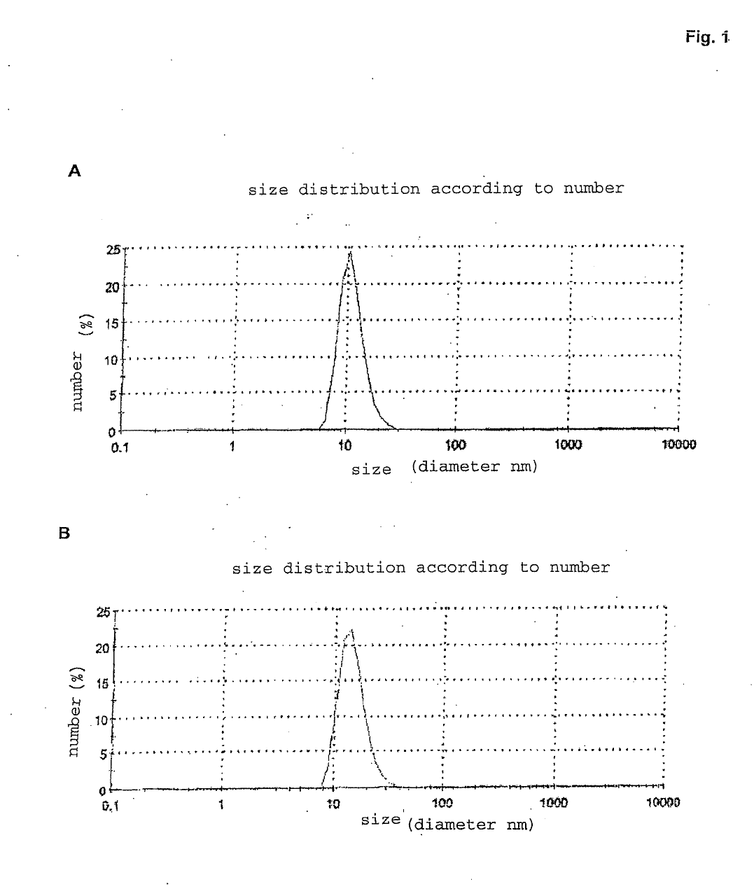

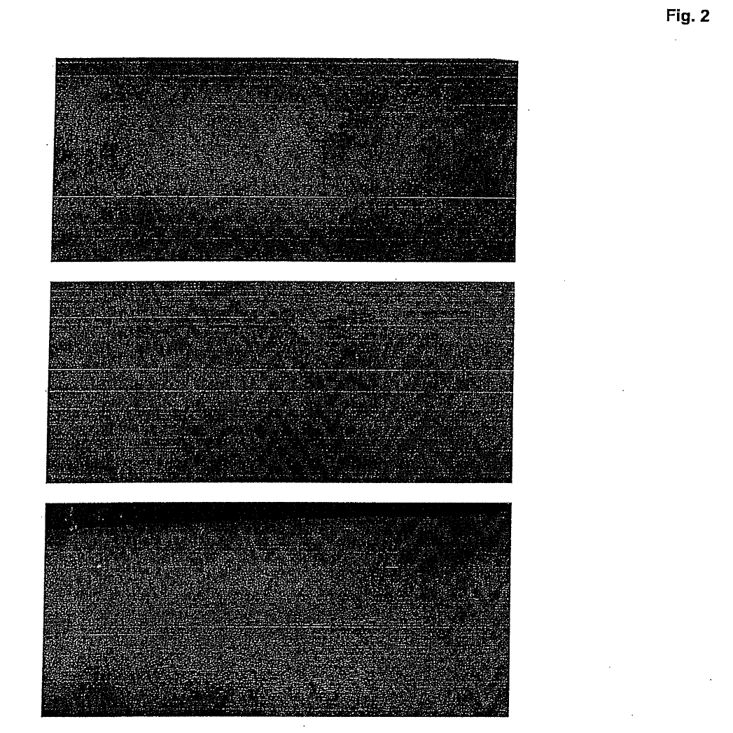

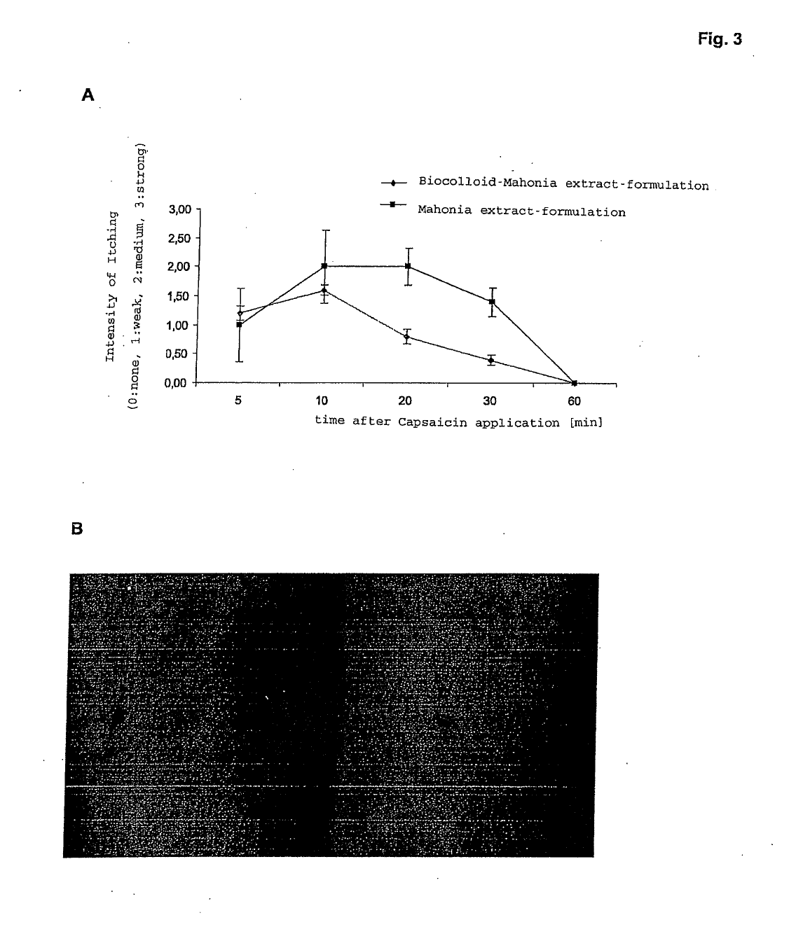

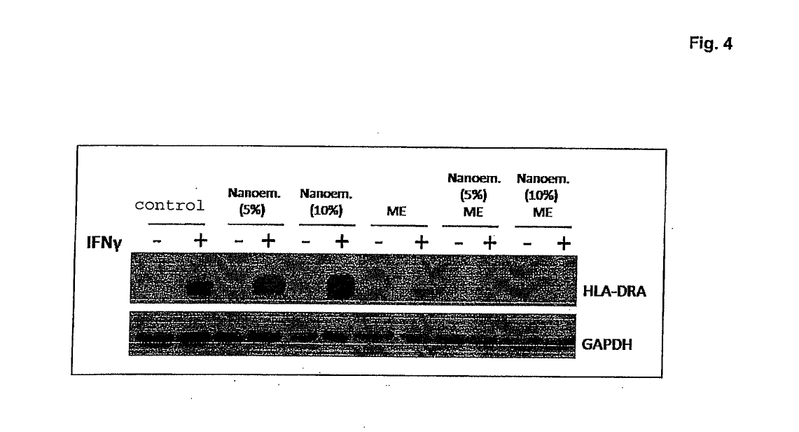

In 15 voluntary test persons with healthy skin acute skin redness was caused at the volar side of the arm at four sides by the strip method of removing horny layers and subsequent treatment with 30% formic acid. About half a minute after the injury, different formulations (ca. 150 mg) comprising an extract from Mahonia aquifolium were applied at three of the four sides and gently rubbed in.

The skin condition was judged visually before and after the injury as well as in regular intervals. 3.5 hours after application of the formulations, a significant redness reduction was observed with three different test persons (from left to right):(1) Mahonia aquifolium formulation with liposomes, (2) Mahonia aquifolium formulation with biocolloids, (3) control without extract from Mahonia aquifolium, (4) Rubisan (homeopathic drug with extract from Mahonia aquifolium in a conventional cream formulation).

FIG. 3 shows the itchiness dilution and the redness reduction after application of a composition according to the invention.

In voluntary test persons with healthy skin 5 μl Capsaicin (50 mM) were applied at the volar sides of both forearms. After approximately 30 seconds, a composition according to the present invention comprising a biocolloid originating from a nanoemulsion and an extract from Mahonia aquifolium were applied to one of the two test skin areas. On the other test skin area, a basically identical formulation comprising an extract from Mahonia aquifolium, but which did not contain nanoemulsion was applied (in each case approximately 150 mg).

(A) The itchiness intensity was described over a period of 60 minutes in regular intervals by subjective evaluation of the test person of a scale from 0 to 3.
(B) The application of Capsaicin caused, besides the itchiness, a strong skin redness which was significantly alleviated 30 minutes after application of the composition according to the invention (right figure) in contrast to the formulation without nanoemulsion (left figure).

FIG. 4 shows the effect of a plant extract from Mahonia aquifolium (ME) on the antigen presentation of skin cells (ceratinocytes).

Skin cells of a human ceratinocyte cell line (HaCaT-cells) were treated with the immunostimulant interferon-γ (IFNγ). By incubation with the immunostimulant the expression of the major histocompatibility complex-II-molecule HLA-DRA (human leukocyte antigen DRA) was induced. The major histocompatibility complex plays an important role in the immunorecognition/antigen presentation.

The expression of HLA-DRA could be significantly reduced by a treatment of the skin cells with an extract from Mahonia aquifolium. This effect was further significantly enhanced by the treatment with a composition according to the invention, which comprised a nanoemulsion (nanoem.) and an extract from Mahonia aquifolium. Incubation with a nanoemulsion without plant extract did not have an effect on the expression of proteins involved in antigen presentation. The determination of the amount of glyceraldehyde-3-phosphate-dehydrogenase (GAPDH) served as a control of the protein application in the different approaches.

### EXAMPLE

### Example 1

### Composition of a Cream Formulation

The quantitative and qualitative composition of the dark-yellow, shiny cream having a pH of 5.5 to 7 is given in the table below.

TABLE 1
Composition of the cosmetic Belixos ®

| | % by weight (based on the total |
|---|---|
| Ingredients | weight of the composition) |
| Water | 35-45 |
| Mahonia aquifolium | 7-15 |
| Glycerol | 2-5 |
| propylene glycol | 1-4 |
| Bis-PEG/PPG-14/14 dimethicone | 1-5 |
| cyclopentasiloxane | 0.3-0.7 |
| nanoemulsion | |
| containing: | |
| Phospholipids | 0.1-3 |
| Polysorbate 80 | 0.5-2 |
| capryl-/capric triglycerides | 0.5-2 |
| isopropyl alcohol | 0.1-3 |
| disodium phosphate dihydrate | 0.001-0.1 |
| sodium dihydrogen phosphate dihydrate | 0.01-0.5 |
| water | 19-50 |
| cetyl alcohol | 0.5-4 |
| sodium carbomer | 0.5-3 |
| phenoxyethanol | 0.1-2 |
| camomile extract | 0.02-1 |
| green tea extract | 0.001-0.01 |
| xanthan gum | 0.1-1 |
| disodium EDTA | 0.01-0.5 |
| tocopheryl acetate | 0.01-0.5 |

1. Pharmaceutical and/or cosmetic composition comprising a biocolloid comprising a nanoemulsion and at least one water-soluble plant extract.

2. Pharmaceutical and/or cosmetic composition according to claim 1, wherein the water-soluble plant extract is an extract from Mahonia aquifolium, an extract from Glycyrrhiza glabra, an extract from Salicornia herbacea, a leaf extract from Plantago lanceolata, a blossom extract from Nympea alba, a bark extract from Cinnamomum zeylanicum or a leaf extract from red wine.

3. Pharmaceutical and/or cosmetic composition according to claim 1, wherein the nanoemulsion comprises a) at least one aqueous component and b) a carrier comprising i) at least one lipophilic component, ii) at least one surfactant and iii) at least one alcohol.

4. Pharmaceutical and/or cosmetic composition according to claim 3, wherein the nanoemulsion comprises the aqueous component in an amount of 50% by weight to 98% by weight based on the total weight of the nanoemulsion.

5. Pharmaceutical and/or cosmetic composition according to claim 3, wherein the nanoemulsion comprises the lipophilic component in an amount of 0.1% by weight to 15% by weight and/or the surfactant in an amount of 1% by weight to 30% by weight and/or the alcohol in an amount of 0.1% by weight to 10% by weight based on the total weight of the nanoemulsion.

6. Pharmaceutical and/or cosmetic composition according to claim 3, wherein the lipophilic component comprises a triglyceride or a mixture of triglycerides.

7. Pharmaceutical and/or cosmetic composition according to claim 3, wherein the surfactant comprises a phospholipid and/or a non-ionic surfactant.

8. Pharmaceutical and/or cosmetic composition according to claim 3, wherein the surfactant comprises a lecithin, in particular soya lecithin and/or a polyoxyethylene surfactant, in particular Polysorbate 80.

9. Pharmaceutical and/or cosmetic composition according to claim 8, wherein the lecithin has a phosphatidyl choline content of at least 80% by weight.

10. Pharmaceutical and/or cosmetic composition according to claim 3, wherein the alcohol comprises at least two carbon atoms.

11. Pharmaceutical and/or cosmetic composition according to claim 10, wherein the alcohol is ethanol or 2-propanol.

12. Pharmaceutical and/or cosmetic composition according to claim 1, wherein the nanoemulsion comprises emulsified particles with an average diameter of 1 nm to 500 nm.

13. Pharmaceutical and/or cosmetic composition according to claim 12, wherein the emulsified particles have an average diameter of less than 100 nm.

14. Pharmaceutical and/or cosmetic composition according to claim 12, wherein the emulsified particles have an average diameter of 10 to 50 nm.

15. Pharmaceutical and/or cosmetic composition according to claim 1, wherein the water-soluble plant extract is obtained from dried components of the particular plant by an extraction method using water and an alcohol.

16. Pharmaceutical and/or cosmetic composition according to claim 1, wherein the water-soluble plant extract is an extract from Mahonia aquifolium and contains isoquinoline alkaloids.

17. Pharmaceutical and/or cosmetic composition according to claim 16, wherein the water-soluble plant extract from Mahonia aquifolium contains berberine.

18. Pharmaceutical and/or cosmetic composition according to claim 1, wherein the nanoemulsion is present in an amount of 20% by weight to 90% by weight and the water-soluble plant extract is present in an amount of 1% by weight to 20% by weight based on the total weight of the composition.

19. Pharmaceutical and/or cosmetic composition according to claim 1, additionally comprising an aqueous component in an amount of 20% by weight to 60% by weight and/or at least one filling agent in an amount of 0.01% by weight to 4% by weight and/or at least one preservative in an amount of 0% by weight to 2% by weight based on the total weight of the composition.

20. Pharmaceutical and/or cosmetic composition according to claim 1, additionally comprising an oxidation-inhibiting agent.

21. Pharmaceutical and/or cosmetic composition according to claim 20, wherein the oxidation-inhibiting agent is of plant origin.

22. Pharmaceutical and/or cosmetic composition according to claim 1, further comprising an anti-inflammatory agent.

23. Pharmaceutical and/or cosmetic composition according to claim 22, wherein the anti-inflammatory agent is of plant origin.

24. Pharmaceutical and/or cosmetic composition according to claim 1, wherein the composition contains 0.1% by weight to 3% by weight phospholipids, 0.5% by weight to 2% by weight triglycerides, 0.5% by weight to 2% by weight polyoxyethylene surfactant, 0.1% by weight to 3% by weight alcohol and 30% by weight to 40% by weight water as a nanoemulsion and 7% by weight to 15% by weight plant extract, in particular from Mahonia aquifolium, 0.5% by weight to 3% by weight filling agent, 0.1% by weight to 2% by weight preservative, 0% by weight to 5% by weight of a further anti-inflammatory agent, 0% by weight to 5% by weight oxidation-inhibiting agent, 0% by weight to 20% by weight further additives and the remainder as water based on the total weight of the composition.

25. Pharmaceutical and/or cosmetic composition according to claim 1, wherein the composition is present in the form of a cream, milk, lotion or a gel, serum, hair tonic or a liquid spray.

26. Pharmaceutical and/or cosmetic composition according to claim 1 for topical application.

27. Pharmaceutical and/or cosmetic composition according to claim 1 for application on the skin.

28. Process for the preparation of a pharmaceutical and/or cosmetic composition according to claim 1, comprising the steps:
a) providing an aqueous component,
b) providing a carrier comprising at least one lipophilic component, at least one surfactant and at least one alcohol, wherein the alcohol comprises preferably at least two carbon atoms,
c) mixing the aqueous component of step a) with the carrier of step b), whereby a nanoemulsion is formed,
d) adding an aqueous plant extract, in particular a water-soluble plant from Mahonia aquifolium to the mixture obtained in step c) prior to and/or after the formation of said nanoemulsion and
e) optionally adding further additives and/or excipients to the mixture obtained in step d).

29. Process according to claim 28, wherein the aqueous plant extract is added to the mixture obtained in step c) after formation of a nanoemulsion.

30. Method for the prevention and/or treatment of skin irritations or skin diseases, comprising administration of an effective amount of a pharmaceutical and/or cosmetic composition according to claim 1 to a subject in need thereof.

31. Method according to claim 30 for the prevention and/or treatment of atopic dermatitis or psoriasis.

32. Method according to claim 30 for the prevention and/or treatment of itching or irritated skin.

33. Method according to claim 30 for the prevention and/or treatment of acne or rosacea.

34. Method according to claim 30 for the prevention and/or treatment of insect stings.
